(19) United States
(12) Patent Application Publication
(10) Pub. No.: US 2026/0106370 A1
(43) Pub. Date: Apr. 16, 2026

(54) REFLECTIVE GROUND SHIELD

(71) Applicant: Insulet Corporation, Acton, MA (US)

(72) Inventors: Ehsan Hajisaeid, Windham, NH (US); Colan Ryan, Escondido, CA (US); Ian McLaughlin, Groton, MA (US); Jeffrey Barnes, Medford, MA (US)

(21) Appl. No.: 19/353,300

(22) Filed: Oct. 8, 2025

Related U.S. Application Data
(60) Provisional application No. 63/706,370, filed on Oct. 11, 2024.

(51) Int. Cl. H01Q 1/52; A61M 5/142; H01Q 19/10

(52) U.S. Cl. CPC H01Q 1/526; A61M 5/14248; H01Q 19/10; A61M 2205/0233; A61M 2205/0238

(57) ABSTRACT

An automated medicament delivery device for automated administration of medicament to a user-body is disclosed. The automated medicament delivery device includes a delivery system, a housing, a printed circuit board (PCB), one or more antennas, an adhesive liner, and a reflective ground shield. The delivery system is configured to deliver medicament to the user-body. The housing is configured to enclose multiple components therein. The PCB is within the housing. The one or more antennas are connected to the PCB. The adhesive liner comprises a flexible substrate and an adhesive configured to secure the housing to a user-body. The reflective ground shield is between the PCB and the adhesive, the reflective ground shield being physically separated from the one or more antennas.

### CROSS-REFERENCE TO RELATED APPLICATIONS

This application claims the benefit under 35 U.S.C. § 119(e) of U.S. Provisional Patent Application Ser. No. 63/706,370, filed Oct. 11, 2024, the disclosure of which is hereby incorporated herein in its entirety by this reference.

### TECHNICAL FIELD

This disclosure relates generally to automated medicament administration. Some embodiments relate to a reflective ground shield for automated medicament delivery devices.

### BACKGROUND

Automated medicament delivery devices (AMD, e.g., Automated Insulin Delivery (AID) device, without limitation) are often used to administer medicaments from a reservoir of the AMD to the body of a patient via a cannula inserted into the body to treat medical conditions (e.g., Type 1 Diabetes, without limitation).

The AMD often communicates with other devices, such an analyte sensor and a controller (e.g., a handheld electronic computing device, such as a mobile device or dedicated handheld controller).

Reliable communications between the AMD and other devices may be challenging, given the optimization of packaging space within the AMD and the fixed location of the AMD on a user-body during use. This fixed location results in the antenna of the AMD being close to the surface of the skin of the user-body, which may result in performance degradation of the antenna. The fields from the antenna may extend into the lossy tissue of the user-body, which may lower the efficiency and gain of the antenna. This degradation may impede communication of the AMD with other devices. Further, the high permittivity of human tissue may affect the impedance matching of the antenna so that the strength of the signal between the antenna and radio chip is reduced, which may further reduce an effective range of the antenna. Any degradation and range limitations caused by the proximity of the antenna to the user-body may be apparent during both transmitting and receiving operations of the AMD.

### BRIEF SUMMARY

In one or more illustrative embodiments, the present disclosure provides an automated medicament delivery device for automated administration of medicament to a user-body. The automated medicament delivery device includes a delivery system, a housing, a printed circuit board (PCB), one or more antennas, an adhesive liner, and a reflective ground shield. The delivery system is configured to deliver medicament to the user-body. The housing may be configured to enclose multiple components therein, and the PCB may reside within the housing, and the one or more antennas are connected to the PCB. The adhesive liner may comprise a flexible substrate and an adhesive configured to secure the AMD device to a user-body. A reflective ground shield may reside between the PCB and the adhesive, the reflective ground shield being physically separated from the one or more antennas.

In one or more illustrative embodiments, the present disclosure provides a shielded liner for a wearable device. The wearable device includes one or more antennas. The shielded liner includes an adhesive liner and a reflective ground shield. The adhesive liner includes a flexible substrate and an adhesive on a surface of the flexible substrate. The reflective ground shield may include a ground shield material fixed, directly or indirectly, to the flexible substrate.

In one or more illustrative embodiments, the present disclosure provides a wearable device. The wearable device includes a housing, a printed circuit board (PCB), one or more antennas, and a shielded liner. The PCB may reside within the housing, and the one or more antennas are connected to the PCB. The shielded liner may be connected to or printed on the housing. The shielded liner includes a reflective ground shield with a ground shield material fixed, directly or indirectly, to the housing.

### BRIEF DESCRIPTION OF THE DRAWINGS

For a detailed understanding of the disclosure, reference should be made to the following detailed description, taken in conjunction with the accompanying drawings, in which like elements have generally been designated with like numerals, and wherein:

FIG. 1 is a schematic diagram illustrating an automated medicament delivery device;

FIG. 2 is a block diagram of a medicament delivery system for controlled administering of medicament, in accordance with one or more embodiments;

FIG. 3 is a perspective view of a portion of an automated medicament delivery device including the adhesive liner, the reflective ground shield, the printed circuit board (PCB), and the one or more antennas, in accordance with one or more embodiments;

FIG. 4 is a perspective view of the portion of the automated medicament delivery device of FIG. 3 illustrating embodiments of the reflective ground shield;

FIG. 5 is a perspective view of the portion of the automated medicament delivery device of FIG. 3 illustrating embodiments of the reflective ground shield;

FIG. 6 is a perspective view of the portion of the automated medicament delivery device of FIG. 3 illustrating embodiments of the reflective ground shield;

FIG. 7 is a cross-sectional view of a shielded liner of the automated medicament delivery device of FIG. 3 illustrating embodiments of the shielded liner;

FIG. 8 is a cross-sectional view of a shielded liner of the automated medicament delivery device of FIG. 3 illustrating embodiments of the shielded liner;

FIG. 9 is a cross-sectional view of a portion of the automated medicament delivery device including an embodiment of the adhesive liner and the reflective ground shield of FIG. 3;

FIG. 10 is a cross-sectional view of a portion of the automated medicament delivery device including an embodiment of the adhesive liner and the reflective ground shield of FIG. 3;

FIG. 11 is a performance plot illustrating gain and efficiency of an automated medicament delivery device without a reflective ground shield;

FIG. 12 is a performance plot illustrating gain and efficiency of an automated medicament delivery device with an embodiment of a reflective ground shield; and

FIG. 13 is a performance plot illustrating gain and efficiency of an automated medicament delivery device with another embodiment of a reflective ground shield.

### DETAILED DESCRIPTION

In various embodiments, a wearable device (e.g., an automated medicament delivery device, without limitation) includes reflective ground shield positioned between the PCB and the user-body. As will be described in detail below, a reflective ground shield may be integrated into the adhesive liner, positioned on the adhesive liner, positioned on a housing of the wearable device, and/or in other similar locations. The reflective ground shield may extend beyond a footprint of the PCB and one or more antennas of the wearable device (e.g., beyond an edge of the PCB in one or more directions, without limitation). The reflective ground shield may include patterns arranged to define resonant and/or non-resonant structures configured to increase performance of the one or more antennas.

The illustrations presented herein are not actual views of any system, device, or structure, or any component thereof, but are merely idealized representations, which are employed to describe various embodiments.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any system, device, or structure, when utilized in a conventional manner. Furthermore, these terms may refer to an orientation of elements of any system, device, or structure, as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

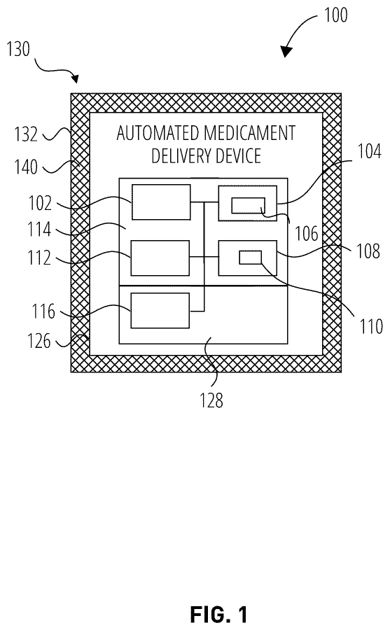

FIG. 1 is a schematic diagram illustrating an automated medicament delivery device (or system) 100 for automated administration of medicament to a user-body, in accordance with one or more embodiments.

In one or more embodiments, the automated medicament delivery device 100 may be capable of one or more operative modes of administration of medicament. Non-limiting examples of the one or more operative modes include: fully automated administration of medicament, partially automated administration of medicament, or manual administration of medicament. In one or more embodiments, the automated medicament delivery device 100 may be capable of alternating between multiple (e.g., two or more, without limitation) operative modes. As a non-limiting example, the automated medicament delivery device 100 may alternate between one or more of: fully automated operation, partially automated operation, and manual operation.

The automated medicament delivery device 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or subcombinations of one or more of the foregoing, without limitation. The automated medicament delivery device 100 may also administer an amount of medicament at least partially based on user inputs (e.g., a user defined bolus amount or details related to a meal consumed or about to be consumed, such as number of carbohydrates, amount of fat, and amount of protein, without limitation).

Non-limiting examples of medicaments administrable by the automated medicament delivery device 100 include: insulin, glucagon-like peptide-1 receptor agonist (GLP-1), glucose-dependent insulinotropic polypeptide (GIP), pramlintide, or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated.

The automated medicament delivery device 100 is configured to administer medicament to a user-body, such as subcutaneously into the user-body, without limitation, in accordance with one or more embodiments. In one or more embodiments, the automated medicament delivery device 100 may offer one or more operative modes for administration of medicament to a user-body. When operating in some of the operative modes, automated medicament delivery device 100 may administer medicament at least partially responsive to analyte values, including without limitation analyte values received from an analyte sensor. The analyte sensor is configured to obtain data related to one or more analytes within the user-body (“analyte data”). The analyte sensor may be an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G6 or G7 manufactured by Dexcom, without limitation).

When operating in some further operative modes, automated medicament delivery device 100 may administer medicament at least partially responsive to user input. When operating some yet further operative modes, automated medicament delivery device 100 may administer medicament at least partially responsive to analyte values and user input. Non-limiting examples of the one or more operative modes offered by automated medicament delivery device 100 include: fully automated administration of medicament, partially automated administration of medicament, or manual administration of medicament. When operating in an operative mode that includes manual administration of medicament, automated medicament delivery device 100 may administer medicament solely in response to a user input (e.g., delivers medicament in response to a user confirmation of delivery of medicament or in response to a user instruction to deliver medicament, without limitation). When operating in an operative mode that includes fully automated administration of medicament, automated medicament delivery device 100 may administer medicament solely in response to analyte values (e.g., delivers medicament in response to one or more analyte values, without limitation). When operating in an operative mode that includes partially automated administration of medicament, automated medicament delivery device 100 may administer medicament in response to analyte values and user input (e.g., delivers medicament in response to a user input and an analyte value, or alternately delivers medicament in response to a user input or in response to analyte values, without limitation). Medicament administration may include administration of a basal amount of medicament regularly delivered over a control interval (e.g., at a determined basal rate, without limitation) to keep analyte levels stable and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and amount of protein, without limitation).

The automated medicament delivery device 100 includes a delivery system 116, one or more processors 102, memory 104, communication equipment 108, a PCB 114, a power source 112, an adhesive liner 132, and a reflective ground shield 140. The automated medicament delivery device 100 may also include a housing 126 configured to enclose the various components of the automated medicament delivery device 100 and a chassis 128 configured to hold or support one or more components (e.g., one or more components of the delivery system 116) of the automated medicament delivery device 100. In one or more embodiments, the automated medicament delivery device 100, or portions thereof, may be a wearable device and may be secured to a user-body. In various embodiments, the delivery system 116 is configured to cause an amount of medicament to move (e.g., flow, without limitation) toward and/or into a user-body.

In various embodiments, delivery system 116 may deliver amounts of medicament at least partially responsive to requests. In various embodiments, instructions 106 of memory 104 may include instructions for determining and generating requests for delivery system 116. In various embodiments, instructions 106 may include instructions for determining one or more amounts of medicament, determining a timing for delivery of one or more amounts of medicament, and for generating one or more requests for delivery system 116 related to the same. When such instructions of instructions 106 are executed by one or more processors 102, the one or more processors 102 determine the amounts of medicament and timing of delivery, generate requests for the delivery system 116 at least partially based on the determined amounts and timing, and provide the requests to delivery system 116.

The communication equipment 108 is configured to facilitate communication (e.g., wireless communication, without limitation) of the automated medicament delivery device 100 with other devices, including without limitation communication between the automated medicament delivery device 100 and the analyte sensor and/or a controller (e.g., a dedicated electronic device, a smart phone, a tablet computer, a wearable device, without limitation). The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various embodiments, the communication equipment 108 includes one or more antennas 110 for the wireless communication. The one or more antennas 110 may be on-board (e.g., printed on the PCB 114, without limitation) or off-board antennas (e.g., connected to the PCB 114, without limitation). In various embodiments, the communication equipment 108 includes an Internet of Things (IOT) Subscriber Identity Module (SIM) card (e.g., a machine-to-machine SIM card, a Universal Integrated Circuit Card, without limitation).

The memory 104, one or more processors 102, and communication equipment 108 may be on and electrically connected via the PCB 114.

The power source 112 is configured to supply power to the delivery system 116 and the various electronic components, such as the one or more processors 102, memory 104, communication equipment 108, and the like. Power source 112 may be, as a non-limiting example, a power storage device (e.g., a battery, without limitation), a power inlet, a power regulator, or combination thereof.

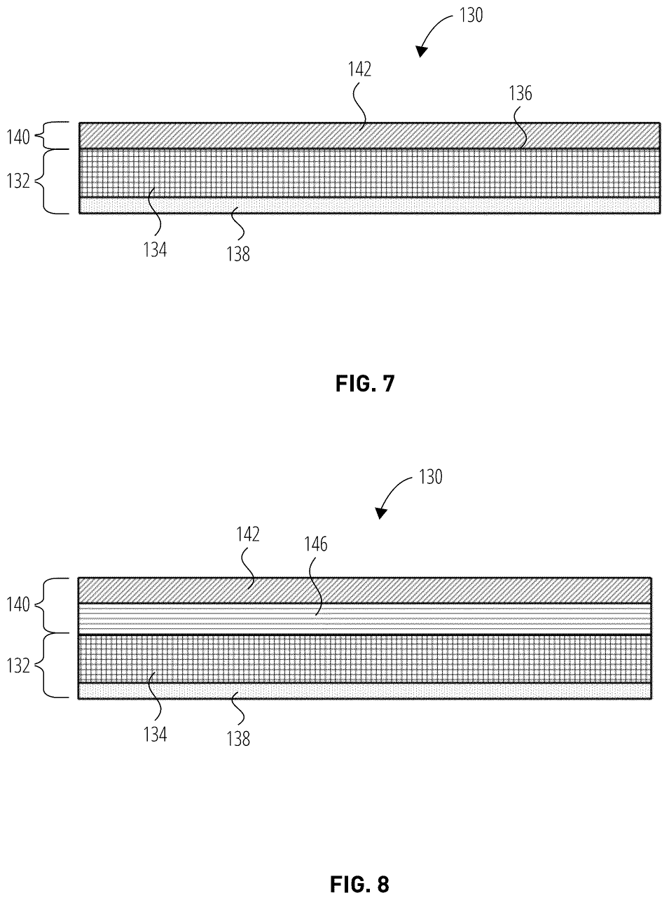

The adhesive liner 132 comprises a flexible substrate 134 (e.g., polyester substrate, sintered fabrics, metallic yarns, or non-woven felts, without limitation) and an adhesive 138 (refer to FIGS. 7 and 8). The adhesive liner 132 is configured to secure the automated medicament delivery device 100 to the user-body. The adhesive liner 132 may be secured to the housing 126 via the adhesive that adheres the flexible substrate to the user-body. The flexible substrate may be joined to the housing 126.

The reflective ground shield 140 is configured to be positioned between the PCB 114 and the user-body while the automated medicament delivery device 100 is adhered to the user-body. In various embodiments, the reflective ground shield 140 is between the PCB 114 and the adhesive 138 of the adhesive liner 132. In some of these various embodiments, the reflective ground shield 140 is on a surface of the adhesive liner 132 opposite the adhesive 138. The reflective ground shield 140 is physically separated from (i.e., contactless/not connected to) the one or more antennas 110, the PCB 114 and other components positioned within the housing 126. In various embodiments, the reflective ground shield 140 covers at least a portion of the adhesive liner 132. The reflective ground shield 140 may extend laterally beyond a footprint of the PCB 114 (e.g., beyond an edge of the PCB in one or more directions, without limitation), the one or more antennas 110, and the housing 126. In various embodiments, the ground shield 140 may extend to about to an outer perimeter of the adhesive liner 132 and in some embodiments may extend beyond the outer perimeter of the adhesive liner 132.

With the reflective ground shield 140 separated from the one or more antennas 110, the ground shield 140 may not cancel the radiated signal thereof. Further, the reflective ground shield 140 extending beyond the edges of the PCB 114 and the one or more antennas 110, the reflective ground shield 140 may act as an effective shield and prevent the one or more antennas 110 from interacting with the user-body. The reflective ground shield 140 defines a ground plane shield that may enhance antenna performance, while mitigating any deleterious effects from the user-body.

In various embodiments, automated medicament delivery device 100 includes a shielded liner 130 that includes the adhesive liner 132 and the reflective ground shield 140. The shielded liner 130 may be formed and joined to the housing 126.

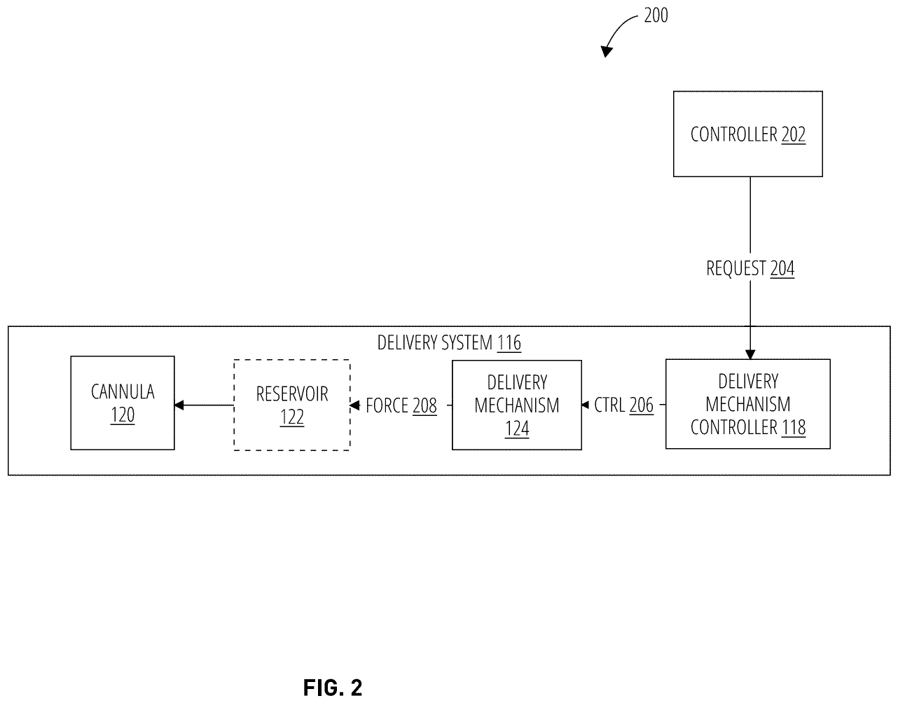

FIG. 2 is a block diagram of an automated medicament delivery system 200 for controlled administration of medicament to a user-body, in accordance with one or more embodiments.

Automated medicament delivery system 200 includes a delivery system 116 and an integrated controller 202 (“controller 202”). Delivery system 116 includes delivery mechanism controller 118, delivery mechanism 124, cannula 120, and reservoir 122. The reservoir 122, which holds the medicament, may be configured as a permanent fixture within the device or as a replaceable component, as a non-limiting example based on user needs or medicament refill practices. In FIG. 2, the block representing the reservoir 122, which stores the medicament, is outlined in dashed lines to indicate it may be either permanently within delivery system 116 or replaceable by a user within delivery system 116.

The controller 202 is configured to manage automated medicament delivery device 100 and, more generally, administration of medicament to a user-body. In one or more embodiments, controller 202 may be implemented by instructions 106 and one or more processors 102 of the automated medicament delivery device 100 of FIG. 1.

In various embodiments, controller 202 and delivery system 116 may be realized in different devices (e.g., controller 202 may be realized in a physically different device (or devices)) than delivery system 116 is realized, or in the same device. When realized in different devices, functionality of controller 202 and delivery system 116 may be implemented, at least in part, by respective memory and one or more processors of their respective devices. When realized in a same device, functionality of controller 202 and delivery system 116 may be implemented, at least in part, by memory and one or more processors, respective memory and respective one or more processors, or a combination thereof. Non-limiting examples of devices in which controller 202, or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 202 may be configured to receive analyte data (e.g., from the analyte sensor, without limitation) including analyte values. In one or more embodiments, controller 202 may determine information about analytes within a user-body at least partially based on analyte data, for example, amounts, trends, distributions, without limitation. The controller 202 may analyze information about analytes in a user-body and may present the information and/or analysis to a patient, caregiver, or healthcare provider, as a non-limiting example, via an application (e.g., executing on a personal computer, smart phone, cloud server, or combinations thereof).

In various embodiments, the controller 202 may be configured to receive information from inputs from the patient or a caregiver (e.g., when the patient ate a meal or when the patient exercised, without limitation), and inputs from other electronic devices (e.g., information from a smartwatch, without limitation) and to utilize such information (e.g., process such information utilizing a control algorithm, without limitation) as discussed herein. For example, in various embodiments, controller 202 may utilize some or a totality of such information to determine amounts of medicament to administer and timing of administration of medicament. Further, controller 202 may also be configured to determine requests, including request to administer dose 204, and send those requests to the automated medicament delivery device 100.

In various embodiments, controller 202 may be configured to determine a target dose amount to administer to a user of medicament delivery system 200. Controller 202 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), doses of fast-acting or rapid-acting insulin, a current glucose value, and derivatives thereof without limitation. The timing and target dose amounts associated with requests generated by controller 202 may be governed by one or more control algorithms, discussed below.

Controller 202 may send a request to administer dose to delivery system 116, and more specifically, delivery mechanism controller 118.

The cannula 120 is insertable into a user-body (e.g., with a tip thereof positioned subcutaneously, without limitation) and is configured to provide medicament to a user-body (e.g., subcutaneously into the user-body, without limitation).

The reservoir 122 is configured to store and retain a medicament therein. As a non-limiting example, the reservoir 122 may be a hollow body, a flexible pouch, a chamber, a vial, without limitation. In various embodiments, reservoir 122 is a fluid reservoir for holding medicament and may be, as a non-limiting example, formed from the walls of a cartridge. In the cartridge example, delivery system 116 may include a chamber (i.e., a space or region defined within delivery system 116) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 122 will be greater in a pre-filled state than the volume in an exhausted state. Additionally or alternatively to the cartridge example, delivery system 116 may be a multi-part delivery device where one of the two parts includes the reservoir 122 and the other one of the two parts includes the delivery mechanism controller 118. The other one of the two parts may optionally further include controller 202. Either one of the two parts may optionally include delivery mechanism 124 (e.g., a piston pump or a reciprocating pump, without limitation). The one of the two parts that includes reservoir 122 may be disposable (i.e., a “disposable part”) and configured to be removably secured to the other part of medicament delivery system 200. When reservoir 122 is exhausted, the disposable part may be removed and a replacement part, including a reservoir 122 optionally in a pre-filled state, may be installed.

Delivery mechanism 124 is configured to urge fluid in reservoir 122 toward an interface for dispensing fluid (interface not shown). In various embodiments, delivery mechanism 124 may be positioned adjacent to reservoir 122. The delivery mechanism 124 is configured to cause an amount of the medicament to be administered to the user-body by causing the amount to flow from the reservoir 122 toward and into a user-body via cannula 120, which is in fluidic communication with the reservoir 122. In various embodiments, delivery mechanism 124 may utilize any suitable mechanism to generate positive displacement or negative displacement to transfer amounts of medicament from reservoir 122 toward cannula 120 and a user-body.

For example, delivery mechanism 124 may apply a force to a piston free to move within reservoir 122, and via such a force, move the piston in a direction that urges fluid in reservoir 122 toward the aforementioned interface. In one or more examples, delivery mechanism 124 may include an electrical motor (e.g., an AC or DC motor) that produces a force to, directly or indirectly, move the piston to perform a delivery action. A delivery action dispenses at a predetermined rate or volume of medicament (i.e., a predictable amount of fluid over a predictable duration of time). The delivery mechanism 124 may be capable of multiple rates of delivery, and in one or more examples, may be preconfigured to use a same rate of delivery all the time, or, in some cases, may be provided discretion to determine a rate of delivery consistent with a target dose amount included with a request.

Such an electric motor may be a current controlled electric motor, voltage controlled electric motor, pulse-width controlled electric motor, or combination or sub combination thereof. Such an electronic motor may be directly or indirectly digitally controlled. The control signal 206 may be determined and generated by delivery mechanism controller 118 to correspond to a delivery action. A control signal 206 may also be referred to herein as a “command 206” or an “instruction 206.”

Delivery mechanism controller 118 may generate control signal(s) 206 corresponding to one or more delivery actions at least partially based on a request to administer dose 204 received from controller 202. Control signal 206 may include first control signals to cause delivery mechanism 124 to generate resultant force 208, and a second, different control signal(s) to cause drive delivery mechanism 124 to not or stop generating force 208. Utilizing control signals 206, delivery mechanism controller 118 may control a length of a duration of time that delivery mechanism 124 produces force 208 and applies it to dispense fluid from reservoir 122, and indirectly, an amount of fluid dispensed from reservoir 122.

When delivery mechanism controller 118 generates control signal(s) 206 in response to a request to administer dose 204 from controller 202, it may generate the control signal(s) 206 at least partially based on a value of a target dose amount included with, or indicated by, request to administer dose 204. One or more delivery actions may be utilized to dispense an amount fluid corresponding to a dose amount determined by controller 202. For example, a fluid amount dispensed according to a delivery action may be less than a dose amount. Generally speaking, the delivery mechanism 124 and delivery system 116 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, delivery mechanism controller 118 may be configured to determine and generate feedback information about delivery actions, such as times of delivery actions and dispensed amounts, without limitation. Feedback information may be generated based on information generated by delivery mechanism 124 or by sensors utilized by delivery mechanism controller 118 to monitor operation of delivery mechanism 124 (sensors not depicted). For example, sensors to monitor mechanical movement, current consumption, a voltage profile of an electric motor, reservoir fluid amount, without limitation, may be utilized. Such information may be logged and provided to and stored at controller 202, without limitation, e.g., for later processing or reading, without limitation. For example, the logs can be processed to determine patterns that may be utilized to determine whether delivery system 116 is operating as expected (e.g., in a predictable manner, without limitation), and if a difference between actual and expected operation exceeds a threshold, delivery mechanism controller 118 may be updated (e.g., firmware, parameters, or both, of delivery mechanism controller 118 may be updated, without limitation) to compensate or correct for the difference. Additionally or alternatively to updating the firmware or parameters, in a multi-part system, one or more parts including delivery mechanism controller 118 or controller 202 may be indicated as needing replacement (e.g., an alarm or alert is generated at delivery system 116, medicament delivery system 200, a mobile device or computer in communication therewith, without limitation).

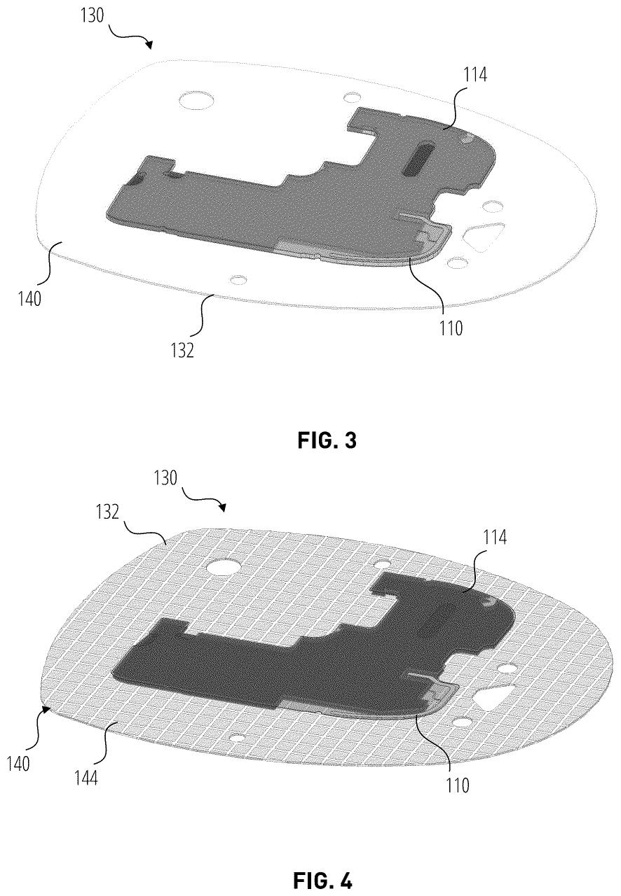

FIG. 3 is a perspective view of a portion of an automated medicament delivery device 100 including the adhesive liner 132, the reflective ground shield 140, the PCB 114, and the one or more antennas 110, in accordance with one or more embodiments. Referring to FIG. 3, in various embodiments, the reflective ground shield 140 comprises a conductive material (e.g., copper, silver, gold, conductive carbon ink, metallic fibers, carbon fibers, Graphene, or carbon nanotube(CNT), without limitation) joined to a surface of the adhesive liner 132. The conductive material may be a solid metal (e.g., metal foil or similar structure, without limitation) and may be joined to the adhesive liner 132 by pasting, printing, and the like. The solid metal may substantially cover the flexible substrate 134 of the adhesive liner 132. In various embodiments, the reflective ground shield 140 is joined to the flexible substrate 134 by screen printing using one or more inks (e.g., silver, carbon ink, without limitation) or by a direct printing method (e.g., Inkjet, gravure, flexographic, Aerosol jet printing, without limitation).

FIG. 4 is a perspective view of the portion of the automated medicament delivery device 100 of FIG. 3 illustrating an embodiment of the reflective ground shield 140. Referring to FIG. 4, the reflective ground shield 140 comprises a pattern 144 of conductive material (e.g., copper, silver, without limitation) on the adhesive liner 132. The pattern 144 may comprise a paste of the conductive material printed on a surface of the adhesive liner 132 (e.g., the surface of the adhesive liner 132 opposite the adhesive, without limitation). In various embodiments, the pattern 144 comprises a grid with the grid lines thereof formed of the conductive material. The pattern 144 may be configured to define a ground plane.

The spacing of the grid lines may be optimized based on a wavelength of the operating frequency of the one or more antennas 110. In various embodiments, the spacing between the grid lines is less than λ₀/10 (e.g., from one-quarter inch to one-half inch, without limitation) where λ₀ is the wavelength of the operating frequency (e.g., 2.45 GHz, without limitation) of the one or more antennas 110.

The spacing and the width of the grid lines may also be optimized for other antenna parameters (e.g., gain, bandwidth, without limitation).

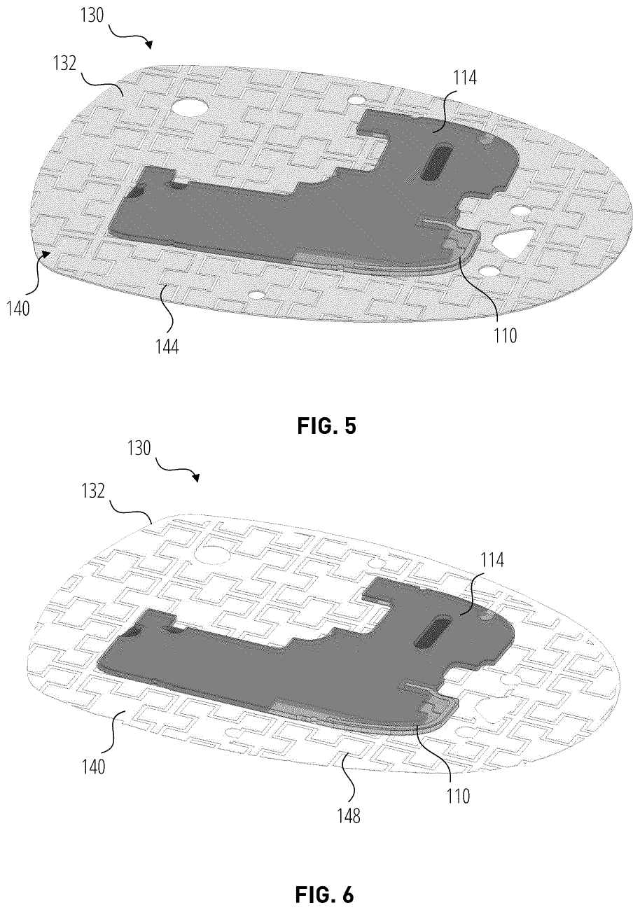

FIG. 5 is a perspective view of the portion of the automated medicament delivery device 100 of FIG. 3 illustrating an embodiment of the reflective ground shield 140. Referring to FIG. 5, in various embodiments, the pattern 144 comprises lines of the conductive material that define an array of resonant structures, such as a metasurface, without limitation. A metasurface is a type of engineered surface that is formed from an array of resonant structures (often sub-wavelength in size), and that can manipulate electromagnetic waves in a controlled manner. A metasurface provides more sophisticated control over the electromagnetic waves. Instead of just reflecting the waves, the metasurface may manipulate the phase and amplitude of the reflected signal. The array may be configured to reflect the signal from the one or more antennas 110 with a specified phase and amplitude, which may either boost antenna gain or reduce the required spacing between the adhesive liner 132 and the one or more antennas 110 without decreasing gain. The pattern 144 may define multiple resonances configured to increase a bandwidth of the one or more antennas 110 and/control the frequency response of the one or more antennas 110. In various embodiments, the pattern 144 includes multiple resonant structures that form current loops (which are inductive) and closely spaced wire sections (which are capacitive). In some of these various embodiments, the pattern 144 includes one or more resonant structures including a bandstop configuration, meaning they are designed to block or attenuate specific frequency bands. Each of the resonant structures may include a different shape for bandstop configurations. Examples of resonant structures include, but are not limited to, a bandstop filter, Jerusalem cross, woodpile, and dogbone structures.

FIG. 6 is a perspective view of the portion of the automated medicament delivery device 100 of FIG. 3 illustrating an embodiment of the reflective ground shield 140. Referring to FIG. 6, in various embodiments, the reflective ground shield 140 includes a layer of conductive material with slots 148 formed therein. The slots may define one or more structures with a bandpass configuration. Each of the resonant structures may include a different shape for bandpass configurations. The layer of conductive material may substantially cover the flexible substrate 134 of the adhesive liner 132.

FIG. 7 is a cross-sectional view of a shielded liner 130 of the automated medicament delivery device 100 of FIG. 3 illustrating embodiments of the shielded liner 130. Referring to FIG. 7, The reflective ground shield 140 includes a ground shield material 142 that is fixed, directly or indirectly, to the flexible substrate 134 (e.g., on, embedded, or within the flexible substrate 134 or on embedded, or within another substrate attached to the flexible substrate 134, without limitation) or integrated into a material of the flexible substrate 134 (e.g., a metallic yarn, without limitation). In various embodiments, the ground shield material 142 is located directly on the flexible substrate 134 of the adhesive liner 132 on the surface 136 of the adhesive liner 132 opposite/distal to the adhesive 138. The ground shield material 142 may be any of the materials for the reflective ground shield 140 disclosed herein.

In various embodiments the combination of the adhesive liner 132 and reflective ground shield 140 comprise a metallized polyester film with a thin coating of the ground shield material 142 (e.g., aluminum, without limitation) deposited onto the flexible substrate 134 (e.g., a polyester film, such as a Mylar® film, without limitation). The flexible substrate 134 may be produced with a rough surface to promote adhesion to the skin of the user-body. The layer of conductive material may substantially cover the flexible substrate 134 of the adhesive liner 132.

FIG. 8 is a cross-sectional view of a shielded liner 130 of the automated medicament delivery device 100 of FIG. 3 illustrating embodiments of the shielded liner 130. Referring to FIG. 8, in various embodiments, the reflective ground shield 140 includes a flexible substrate 146 and the ground shield material 142. In these embodiments, the ground shield material 142 is joined to or printed onto the flexible substrate 146. The flexible substrate 146 of the reflective ground shield 140 is attached to the flexible substrate 134 of the adhesive liner 132, for example, by a heat stack method, ultrasonic welding, or a conductive adhesive, without limitation. The flexible substrate 146 may include a flexible circuit board material (e.g., polyimide film (such as Kapton®), liquid crystal polymers (LCP), or low-density polyethylene (LDPE), or a polyester film without limitation).

In various embodiments, the reflective ground shield 140 includes a metallized polyester film with a coating of the ground shield material 142 (e.g., aluminum, without limitation) deposited onto the flexible substrate 146 (e.g., a polyester film, such as a Mylar® film, without limitation). The flexible substrate 146 is then joined to the flexible substrate 134 opposite the adhesive 138.

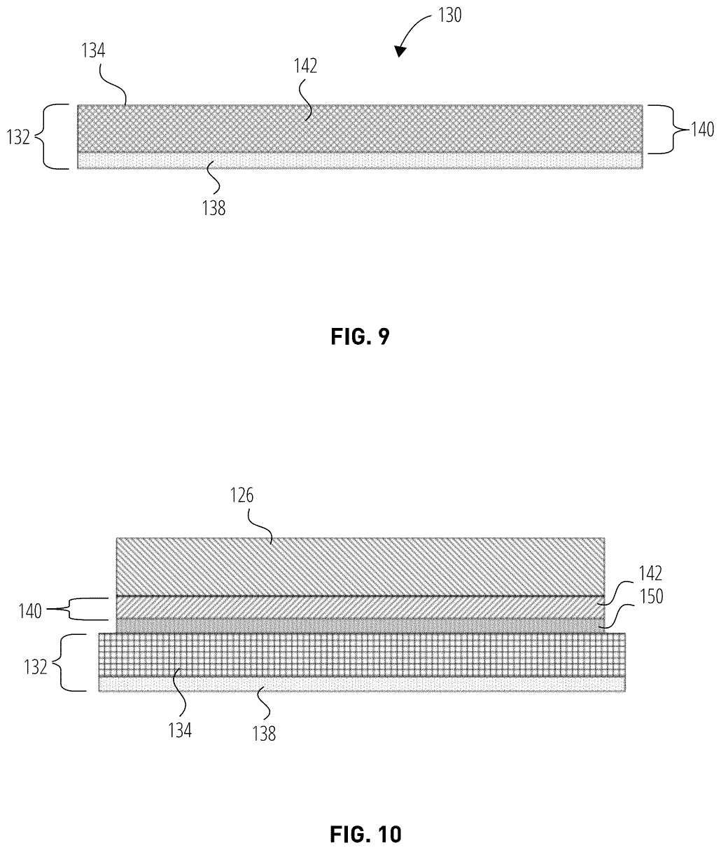

FIG. 9 is a cross-sectional view of a portion of the automated medicament delivery device 100 including an embodiment of the adhesive liner 132 and the reflective ground shield 140 of FIG. 3. Referring to FIG. 9, in various embodiments, the ground shield material 142 is integrated into a flexible substrate, such as the flexible substrate 134 or a separate flexible substrate 146. The ground shield material 142 may include fibers or strands (e.g., carbon fibers or metallic strands, without limitation) that are interspersed within a woven flexible substrate. The woven flexible substrate may include traditional textile materials.

FIG. 10 is a cross-sectional view of a portion of the automated medicament delivery device 100 including an embodiment of the adhesive liner 132 and the reflective ground shield 140 of FIG. 3. Referring to FIG. 10, in various embodiments, the reflective ground shield 140 is located on a bottom side of the housing 126, the bottom side being the side situated closest to the user-body while the automated medicament delivery device 100 is attached thereto. The ground shield material 142 may be insert-molded with the housing 126 or fabricated separately from the housing 126 and joined thereto. More rigid and higher-conductivity materials may be used for ground shield materials 142 on the housing 126. The adhesive liner 132 may be connected to the reflective ground shield located on a bottom side of the housing 126 via an adhesive 150.

The reflective ground shield 140 may be joined to the flexible substrate 134, formed on the flexible substrate 134, integrated within the flexible substrate 134, integrated within a separate flexible substrate 146, joined to the housing 126, insert-molded with the housing 126, or any combination thereof.

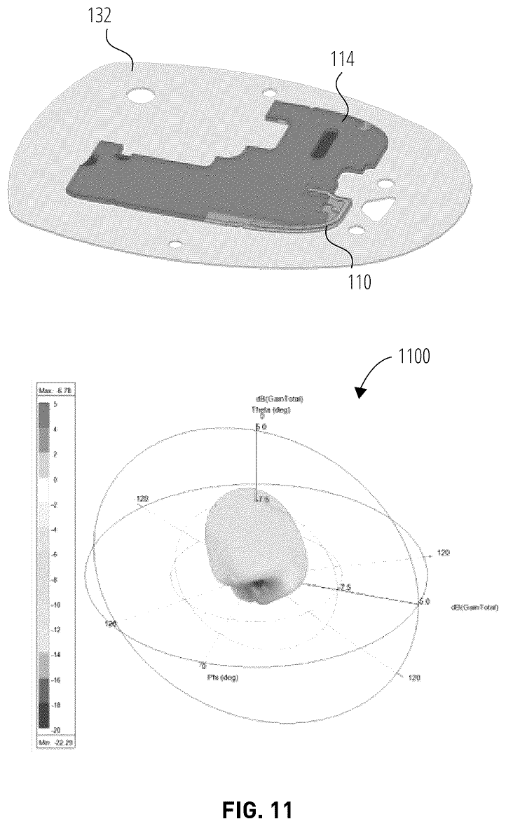

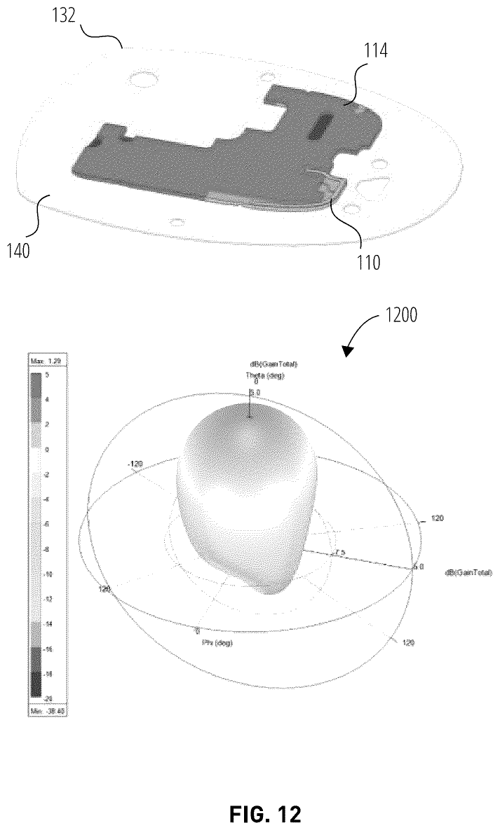

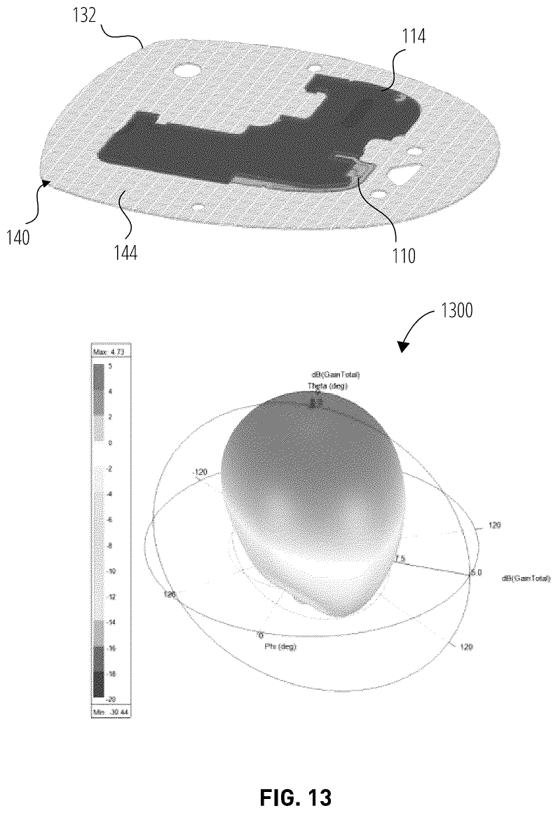

FIG. 11 is a performance plot 1100 illustrating gain and efficiency of an automated medicament delivery device without a reflective ground shield FIG. 12 is a performance plot 1200 illustrating gain and efficiency of an automated medicament delivery device with an embodiment of a reflective ground shield. FIG. 13 is a performance plot 1300 illustrating gain and efficiency of an automated medicament delivery device with another embodiment of a reflective ground shield. Referring to FIGS. 11-13, the max gain and efficiency improves from an automated medicament delivery device without a reflective ground shield (FIG. 11) to an automated medicament delivery device with a reflective ground shield (FIGS. 12 and 13).

For example, in performance plot 1100, for the automated medicament delivery device without the reflective ground shield, the max gain is −6.78 dB and the efficiency is 5.2%, while the performance plot 1200 for an automated medicament delivery device with the reflective ground shield, the max gain is 1.29 dB and the efficiency is 23.9%. Further, performance plot 1300 illustrates that by optimizing the reflective ground shield utilizing, for example, a gridded layout, the max gain and efficiency can be further improved. Indeed, in performance plot 1300, the max gain is 4.73 dB and the efficiency is 50.3%.

While embodiments of the reflective ground shield discussed herein are described in connection with automated medicament delivery systems and devices, such a reflective ground shield may be utilized with other wearable devices including an antenna (e.g., an analyte sensor, an automated medicament delivery systems or devices, a smart watch, a heart monitor, a Ubiqvue biosensor, or devices with modifications that would be apparent to a person having ordinary skill in the art, without limitation).

Non-limiting illustrative embodiments of this disclosure may include:

### Embodiment 1

An automated medicament delivery device for automated administration of medicament to a user-body, the automated medicament delivery device comprising: a delivery system configured to deliver medicament to the user-body; a housing configured to enclose multiple components therein; a printed circuit board (PCB) within the housing; one or more antennas connected to the PCB; an adhesive liner comprising a flexible substrate and an adhesive configured to secure the housing to a user-body; and a reflective ground shield between the PCB and the adhesive, the reflective ground shield being physically separated from the one or more antennas.

### Embodiment 2

The automated medicament delivery device according to Embodiment 1, wherein the reflective ground shield extends laterally in each direction beyond a footprint of the PCB.

### Embodiment 3

The automated medicament delivery device according to any of Embodiments 1 and 2, wherein the reflective ground shield comprises a conductive material.

### Embodiment 4

The automated medicament delivery device according to any of Embodiments 1 through 3, wherein the conductive material includes at least one material chosen from among copper, silver, gold, conductive carbon ink, metallic fibers, and carbon fibers.

### Embodiment 5

The automated medicament delivery device according to any of Embodiments 1 through 4, wherein the conductive material includes a solid metal substantially covering the flexible substrate of the adhesive liner.

### Embodiment 6

The automated medicament delivery device according to any of Embodiments 1 through 5, wherein the solid metal comprises a metal foil.

### Embodiment 7

The automated medicament delivery device according to any of Embodiments 1 through 6, wherein the reflective ground shield is joined to a surface of the flexible substrate.

### Embodiment 8

The automated medicament delivery device according to any of Embodiments 1 through 7, wherein the reflective ground shield comprises a pattern of conductive material on the flexible substrate.

### Embodiment 9

The automated medicament delivery device according to any of Embodiments 1 through 8, wherein the pattern comprises a grid with grid lines thereof formed of the conductive material.

### Embodiment 10

The automated medicament delivery device according to any of Embodiments 1 through 9, wherein a spacing between the grid lines is less than λ₀/10 where λ₀ is a wavelength of an operating frequency of the one or more antennas.

### Embodiment 11

The automated medicament delivery device according to any of Embodiments 1 through 10, wherein the pattern comprises an array of resonant structures.

### Embodiment 12

The automated medicament delivery device according to any of Embodiments 1 through 11, wherein the array of resonant structures is configured to reflect a signal from the one or more antennas with a specified phase and amplitude.

### Embodiment 13

The automated medicament delivery device according to any of Embodiments 1 through 12, wherein the array of resonant structures defines multiple resonances configured to increase a bandwidth of the one or more antennas.

### Embodiment 14

The automated medicament delivery device according to any of Embodiments 1 through 13, wherein the array of resonant structures includes multiple resonant structures that form current loops.

### Embodiment 15

The automated medicament delivery device according to any of Embodiments 1 through 14, wherein the array of resonant structures includes one or more resonant structures includes a bandstop configuration.

### Embodiment 16

The automated medicament delivery device according to any of Embodiments 1 through 15, wherein the array of resonant structures comprises a metasurface.

### Embodiment 17

The automated medicament delivery device according to any of Embodiments 1 through 16, wherein the reflective ground shield comprises a layer of conductive material defining slots therein.

### Embodiment 18

The automated medicament delivery device according to any of Embodiments 1 through 17, wherein the layer of conductive material and the slots comprise one or more structures with a bandpass configuration.

### Embodiment 19

The automated medicament delivery device according to any of Embodiments 1 through 18, wherein the layer of conductive material substantially covers the flexible substrate of the adhesive liner.

### Embodiment 20

The automated medicament delivery device according to any of Embodiments 1 through 19, wherein the flexible substrate comprises a polyester film and the reflective ground shield comprises a coating of a ground shield material on the polyester film.

### Embodiment 21

The automated medicament delivery device according to any of Embodiments 1 through 20, wherein the reflective ground shield comprises a second flexible substrate and a ground shield material fixed thereto, the second flexible substrate joined to the flexible substrate of the adhesive liner.

### Embodiment 22

The automated medicament delivery device according to any of Embodiments 1 through 21, wherein the second flexible substrate comprises a flexible circuit board material.

### Embodiment 23

The automated medicament delivery device according to any of Embodiments 1 through 22, wherein the second flexible substrate comprises a polyester film and the ground shield material comprises a coating on the polyester film.

### Embodiment 24

The automated medicament delivery device according to any of Embodiments 1 through 23, wherein the ground shield material is integrated into the second flexible substrate.

### Embodiment 25

The automated medicament delivery device according to any of Embodiments 1 through 24, wherein the second flexible substrate comprises a woven flexible substrate and the ground shield material is interspersed within the woven flexible substrate.

### Embodiment 26

The automated medicament delivery device according to any of Embodiments 1 through 25, wherein a ground shield material of the reflective ground shield is integrated into the flexible substrate.

### Embodiment 27

The automated medicament delivery device according to any of Embodiments 1 through 26, wherein the flexible substrate comprises a woven flexible substrate and the ground shield material is interspersed within the woven flexible substrate.

### Embodiment 28

The automated medicament delivery device according to any of Embodiments 1 through 27, wherein the reflective ground shield is located on or within the housing.

### Embodiment 29

The automated medicament delivery device according to any of Embodiments 1 through 28, wherein a ground shield material of the reflective ground shield is insert-molded with the housing.

### Embodiment 30

A shielded liner for a wearable device, the wearable device including one or more antennas, the shielded liner comprising: an adhesive liner including a flexible substrate and an adhesive on a surface of the flexible substrate; and a reflective ground shield comprising a ground shield material fixed, directly or indirectly, to the flexible substrate.

### Embodiment 31

The shielded liner according to Embodiment 30, wherein the ground shield material comprises a conductive material.

### Embodiment 32

The shielded liner according to any of Embodiments 30 and 31, wherein the conductive material includes at least one material chosen from among copper, silver, gold, conductive carbon ink, metallic fibers, and carbon fibers.

### Embodiment 33

The shielded liner according to any of Embodiments 30 through 32, wherein the ground shield material includes a solid metal substantially covering the flexible substrate of the adhesive liner.

### Embodiment 34

The shielded liner according to any of Embodiments 30 through 33, wherein the solid metal comprises a metal foil.

### Embodiment 35

The shielded liner according to any of Embodiments 30 through 34, wherein the reflective ground shield is joined to the surface of the flexible substrate.

### Embodiment 36

The shielded liner according to any of Embodiments 30 through 35, wherein the reflective ground shield comprises a pattern of conductive material on the flexible substrate.

### Embodiment 37

The shielded liner according to any of Embodiments 30 through 36, wherein the pattern comprises a grid with grid lines thereof formed of the conductive material.

### Embodiment 38

The shielded liner according to any of Embodiments 30 through 37, wherein a spacing between the grid lines is less than λ₀/10 where λ₀ is a wavelength of an operating frequency of the one or more antennas.

### Embodiment 39

The shielded liner according to any of Embodiments 30 through 38, wherein the pattern comprises an array of resonant structures.

### Embodiment 40

The shielded liner according to any of Embodiments 30 through 39, wherein the array of resonant structures is configured to reflect a signal from the one or more antennas with a specified phase and amplitude.

### Embodiment 41

The shielded liner according to any of Embodiments 30 through 40, wherein the array of resonant structures defines multiple resonances configured to increase a bandwidth of the one or more antennas.

### Embodiment 42

The shielded liner according to any of Embodiments 30 through 41, wherein the array of resonant structures includes multiple resonant structures that form current loops.

### Embodiment 43

The shielded liner according to any of Embodiments 30 through 42, wherein the array of resonant structures includes one or more resonant structures includes a bandstop configuration.

### Embodiment 44

The shielded liner according to any of Embodiments 30 through 43, wherein the array of resonant structures comprises a metasurface.

### Embodiment 45

The shielded liner according to any of Embodiments 30 through 44, wherein the reflective ground shield comprises a layer of the ground shield material defining slots therein.

### Embodiment 46

The shielded liner according to any of Embodiments 30 through 45, wherein the layer of the ground shield material and the slots comprise one or more structures with a bandpass configuration.

### Embodiment 47

The shielded liner according to any of Embodiments 30 through 46, wherein the flexible substrate comprises a polyester film and the reflective ground shield comprises a coating of the ground shield material on the polyester film.

### Embodiment 48

The shielded liner according to any of Embodiments 30 through 47, wherein the reflective ground shield comprises a second flexible substrate and the ground shield material fixed thereto, the second flexible substrate joined to the flexible substrate of the adhesive liner.

### Embodiment 49

The shielded liner according to any of Embodiments 30 through 48, wherein the second flexible substrate comprises a flexible circuit board material.

### Embodiment 50

The shielded liner according to any of Embodiments 30 through 49, wherein the second flexible substrate comprises a polyester film and the ground shield material comprises a coating on the polyester film.

### Embodiment 51

The shielded liner according to any of Embodiments 30 through 50, wherein the ground shield material is integrated into the second flexible substrate.

### Embodiment 52

The shielded liner according to any of Embodiments 30 through 51, wherein the second flexible substrate comprises a woven flexible substrate and the ground shield material is interspersed within the woven flexible substrate.

### Embodiment 53

The shielded liner according to any of Embodiments 30 through 52, wherein the ground shield material of the reflective ground shield is integrated into the flexible substrate.

### Embodiment 54

The shielded liner according to any of Embodiments 30 through 53, wherein the flexible substrate comprises a woven flexible substrate and the ground shield material is interspersed within the woven flexible substrate.

### Embodiment 55

A wearable device, comprising: a housing; a printed circuit board (PCB) within the housing; one or more antennas connected to the PCB; and a shielded liner connected to the housing, the shielded liner comprising: an adhesive liner including a flexible substrate and an adhesive on a surface of the flexible substrate; and a reflective ground shield comprising a ground shield material fixed, directly or indirectly, to the flexible substrate.

The embodiments described above and illustrated in the accompanying drawings do not limit the scope of the disclosure, which is encompassed by the scope of the appended claims and their legal equivalents. Any equivalent embodiments are within the scope of this disclosure. Indeed, various modifications, in addition to those shown and described herein, such as alternate useful combinations of the elements described, will become apparent to those skilled in the art from the description. Such modifications and embodiments also fall within the scope of the appended claims and equivalents.

What is claimed is:

1. An automated medicament delivery device for automated administration of medicament to a user-body, the automated medicament delivery device comprising:
a delivery system configured to deliver medicament to the user-body;
a housing configured to enclose multiple components therein;
a printed circuit board (PCB) within the housing;
one or more antennas connected to the PCB;
an adhesive liner comprising a flexible substrate and an adhesive configured to secure the housing to a user-body; and
a reflective ground shield between the PCB and the adhesive, the reflective ground shield being physically separated from the one or more antennas.

2. The automated medicament delivery device of claim 1, wherein the reflective ground shield extends laterally beyond a footprint of the PCB.

3. The automated medicament delivery device of claim 1, wherein the reflective ground shield comprises a conductive material.

4. The automated medicament delivery device of claim 3, wherein the conductive material includes at least one material chosen from among copper, silver, gold, conductive carbon ink, metallic fibers, and carbon fibers.

5. The automated medicament delivery device of claim 3, wherein the conductive material includes a solid metal substantially covering the flexible substrate of the adhesive liner.

6. The automated medicament delivery device of claim 5, wherein the solid metal comprises a metal foil.

7. The automated medicament delivery device of claim 1, wherein the reflective ground shield is joined to a surface of the flexible substrate.

8. The automated medicament delivery device of claim 1, wherein the reflective ground shield comprises a pattern of conductive material on the flexible substrate.

9. The automated medicament delivery device of claim 8, wherein the pattern comprises a grid with grid lines thereof formed of the conductive material.

10. A shielded liner for a wearable medical device, the wearable medical device including one or more antennas, the shielded liner comprising:
an adhesive liner including a flexible substrate and an adhesive on a surface of the flexible substrate; and
a reflective ground shield comprising a ground shield material fixed, directly or indirectly, to the flexible substrate.

11. The shielded liner of claim 10, wherein the ground shield material comprises a conductive material.

12. The shielded liner of claim 11, wherein the conductive material includes at least one material chosen from among copper, silver, gold, conductive carbon ink, metallic fibers, and carbon fibers.

13. The shielded liner of claim 10, wherein the ground shield material includes a solid metal substantially covering the flexible substrate of the adhesive liner.

14. The shielded liner of claim 13, wherein the solid metal comprises a metal foil.

15. The shielded liner of claim 10, wherein the reflective ground shield is joined to the surface of the flexible substrate.

16. The shielded liner of claim 10, wherein the reflective ground shield comprises a pattern of conductive material on the flexible substrate.

17. The shielded liner of claim 10, wherein the flexible substrate comprises a polyester film and the reflective ground shield comprises a coating of the ground shield material on the polyester film.

18. The shielded liner of claim 10, wherein the ground shield material of the reflective ground shield is integrated into the flexible substrate.

19. The shielded liner of claim 18, wherein the flexible substrate comprises a woven flexible substrate and the ground shield material is interspersed within the woven flexible substrate.

20. A wearable device, comprising:
a housing;
a printed circuit board (PCB) within the housing;
one or more antennas connected to the PCB; and
a shielded liner connected to the housing, the shielded liner comprising a reflective ground shield comprising a ground shield material fixed, directly or indirectly, to the housing.
